(19) United States
(12) Patent Application Publication
(10) Pub. No.: US 2018/0344292 A1
(43) Pub. Date: Dec. 6, 2018

(54) METHODS AND SYSTEM FOR AUTOMATICALLY ANALYZING A DOPPLER SPECTRUM

(71) Applicant: General Electric Company, Schenectady, NY (US)

(72) Inventors: Kjetil Viggen, Trondheim (NO); Elina Sokulin, Tirat Carmel (IL)

(21) Appl. No.: 15/610,498

(22) Filed: May 31, 2017

(51) Int. Cl. A61B 8/08; A61B 8/06; A61B 8/00

(52) U.S. Cl. CPC A61B 8/488; A61B 8/06; A61B 8/5223; A61B 8/54; A61B 8/5207; A61B 8/461

(57) ABSTRACT

Various methods and systems are provided for automatically analyzing a displayed Doppler spectrum, acquired with an ultrasound system, containing multiple heart cycles of data. As one example, a method may include automatically tracing a spectrum displayed via a user interface of an ultrasound system, the spectrum including data from a plurality of cycles acquired with the ultrasound system; displaying measurement parameters and measurement graphics for the traced spectrum; and automatically updating the displayed measurement parameters for a selected number of cycles out of the plurality of cycles to include in the traced spectrum.

### FIELD

Embodiments of the subject matter disclosed herein relate to ultrasound imaging, and more particularly, to methods and systems for automatically analyzing a Doppler spectrum containing multiple heart cycles of data.

### BACKGROUND

During an ultrasound of the heart, referred to as an echocardiogram, multiple heart cycles (e.g., beats) of ultrasound data are taken. Specifically, a Doppler ultrasound may be used to obtain an image of blood flow through the heart (color Doppler) and/or a blood flow velocity waveform (continuous or pulse wave Doppler) for multiple heart cycles. The Doppler spectrum from the continuous or pulse wave Doppler may be displayed via a user interface or display screen of an ultrasound imaging system. In order to accurately diagnose a patient's heart rhythm, multiple heart cycles of data must be obtained and analyzed (e.g., 3-5 cycles for patients in sinus rhythm and 5-10 cycles for patients with irregular heart rhythms). Data from the acquired cycles may then be individually analyzed and averaged in order to accurately diagnose a patient.

### BRIEF DESCRIPTION

In one embodiment, a method comprises automatically tracing a spectrum displayed via a user interface of an ultrasound system, the spectrum including data from a plurality of cycles acquired with the ultrasound system; displaying measurement parameters and measurement graphics for the traced spectrum; and automatically updating the displayed measurement parameters for a selected number of cycles out of the plurality of cycles to include in the traced spectrum.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasound imaging system according to an embodiment of the invention.

FIG. 2 shows an example display output of a user interface display showing a Doppler ultrasound spectrum acquired with an ultrasound imaging system, according to an embodiment of the invention.

FIG. 3 shows an example display output of a user interface display showing an automatic tracing applied to the Doppler spectrum of FIG. 2, according to an embodiment of the invention.

FIG. 4 shows an example display output of a user interface display showing a selection of an individual cycle out of all traced cycles of the traced Doppler spectrum of FIG. 3, according to an embodiment of the invention.

FIG. 5 shows an example display output of a user interface display showing an automatic tracing applied to the Doppler spectrum of FIG. 2 that is updated in response to rejecting the individual cycle selected in FIG. 4, according to an embodiment of the invention.

FIG. 6 shows an example display output of a user interface display showing final traced cycles of the Doppler spectrum and measurement parameters for each finally selected traced cycle, according to an embodiment of the invention.

FIG. 7 shows an example display output of a user interface showing a worksheet generated for the approved measurement parameters of the approved cycles of the Doppler spectrum of FIG. 6, according to an embodiment of the invention.

FIG. 8 shows a touch panel display including a sensitivity input for adjusting a sensitivity of an automatic tracing of the Doppler spectrum, according to an embodiment of the invention.

FIGS. 9A-9B show a flow chart of a method for automatically analyzing a displayed Doppler spectrum containing multiple heart cycles of data, according to an embodiment of the invention.

FIGS. 10-11 show example display outputs of a user interface display showing an automatic E/A velocity measurement for a Doppler spectrum, according to an embodiment of the invention.

FIG. 12 shows an example display output of a user interface display showing an automatic velocity point measurement for a Doppler spectrum, according to an embodiment of the invention

### DETAILED DESCRIPTION

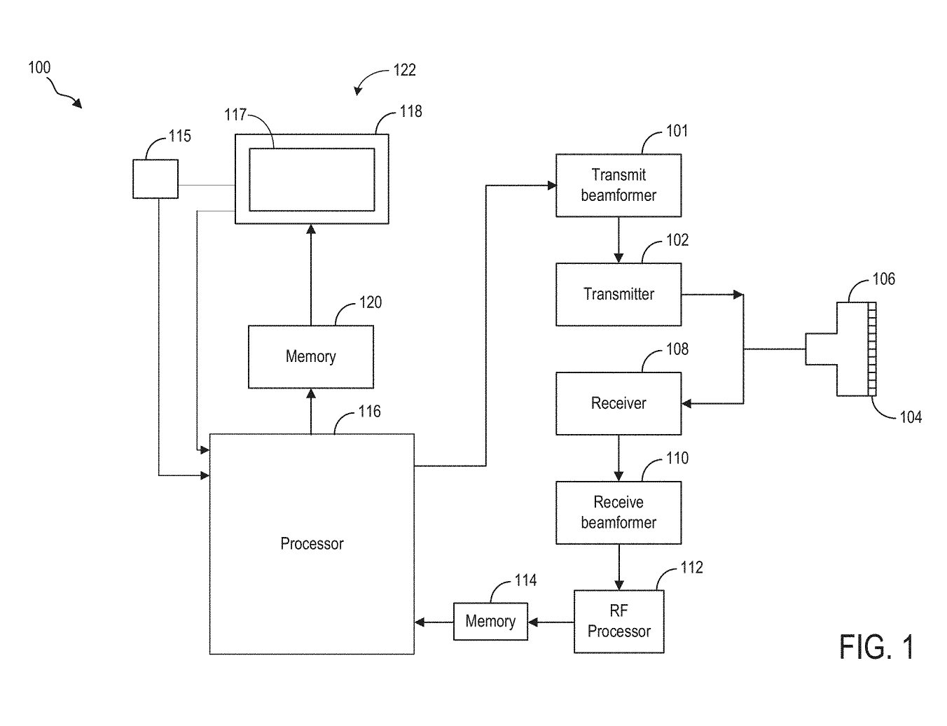

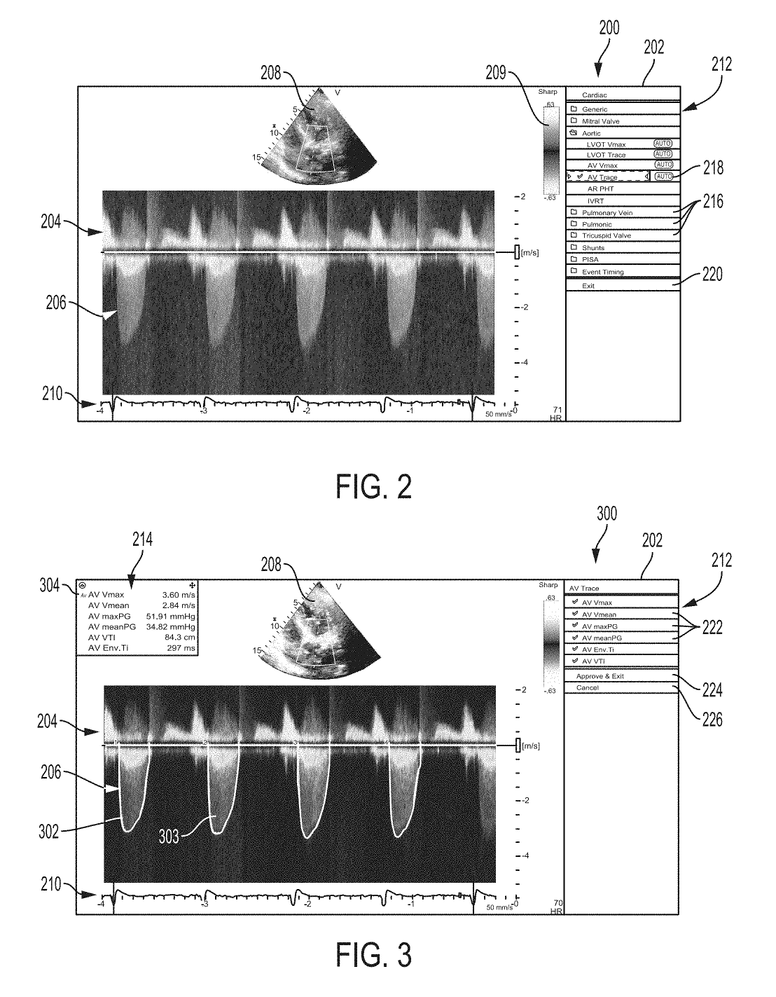

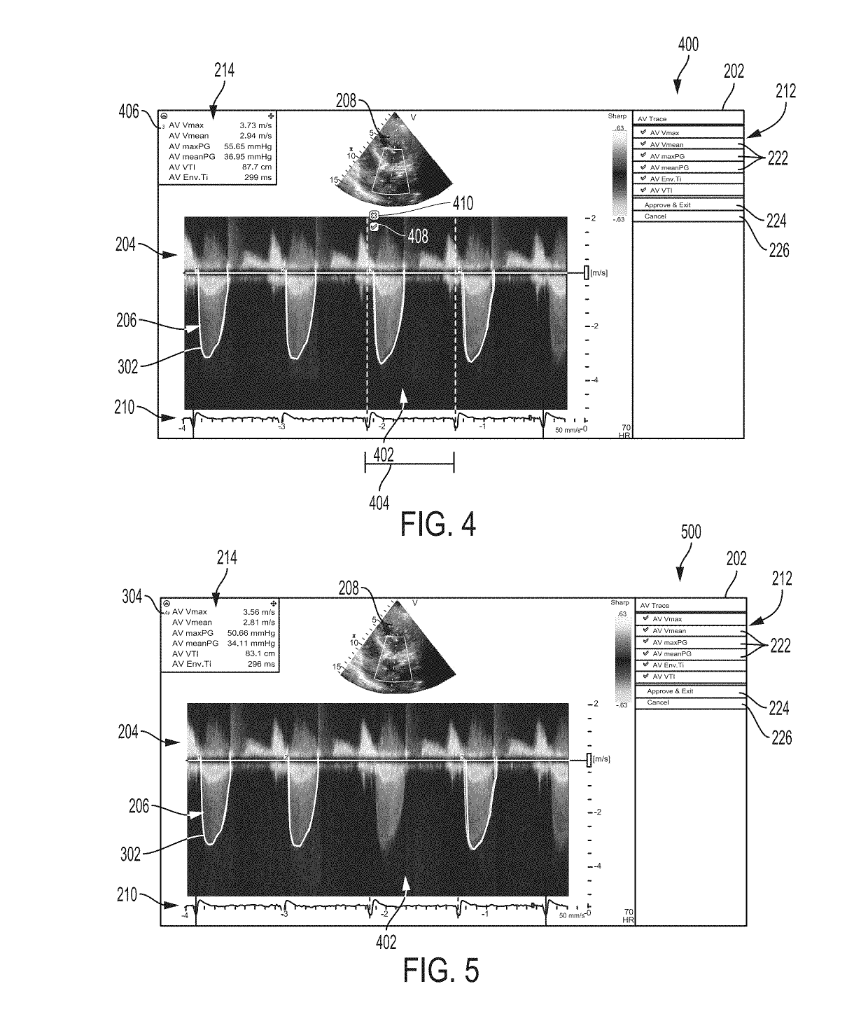

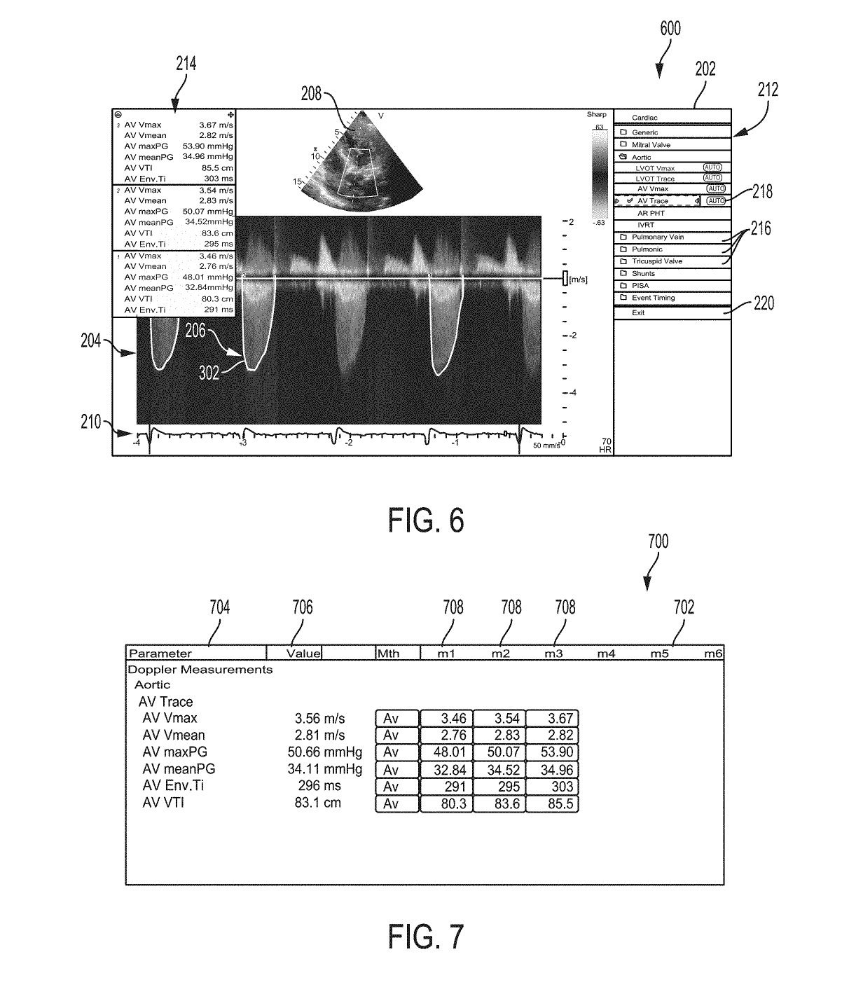

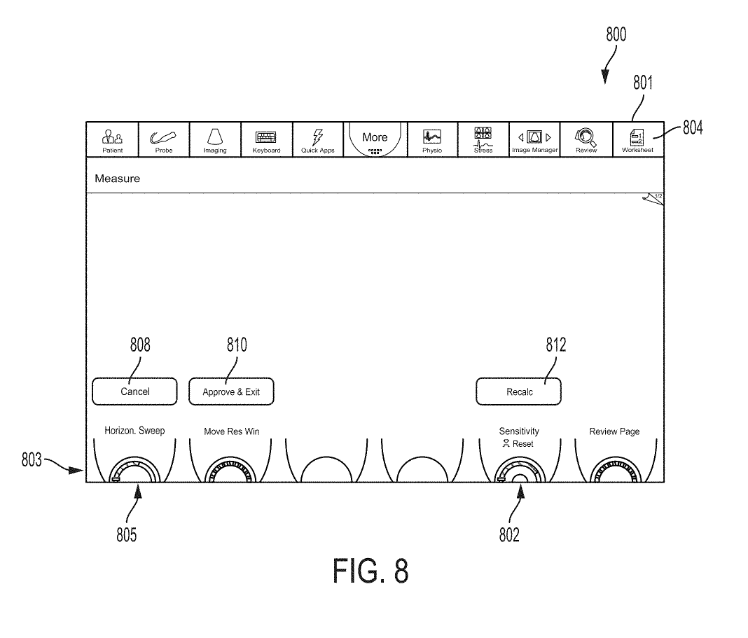

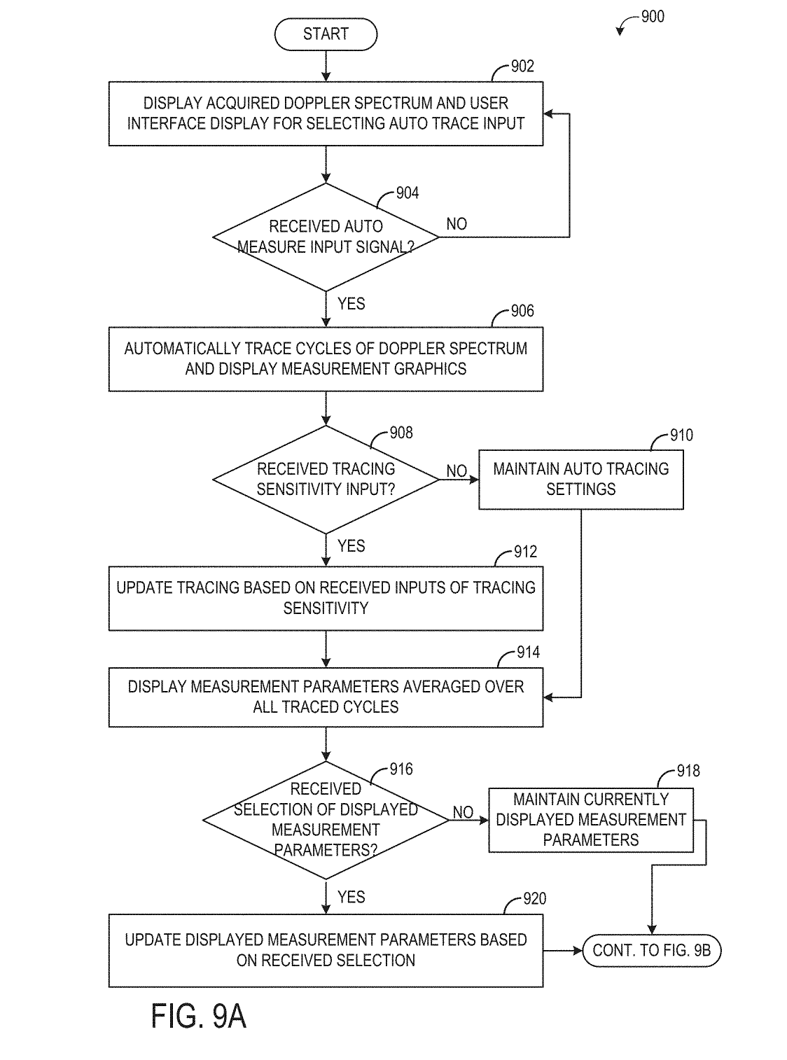

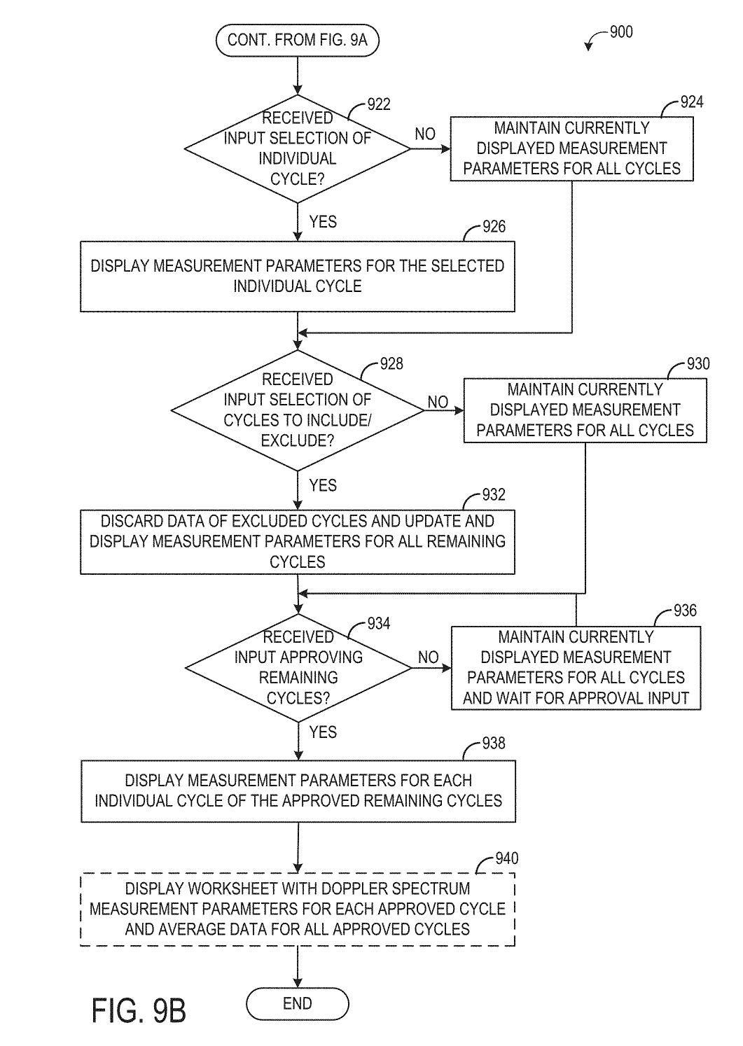

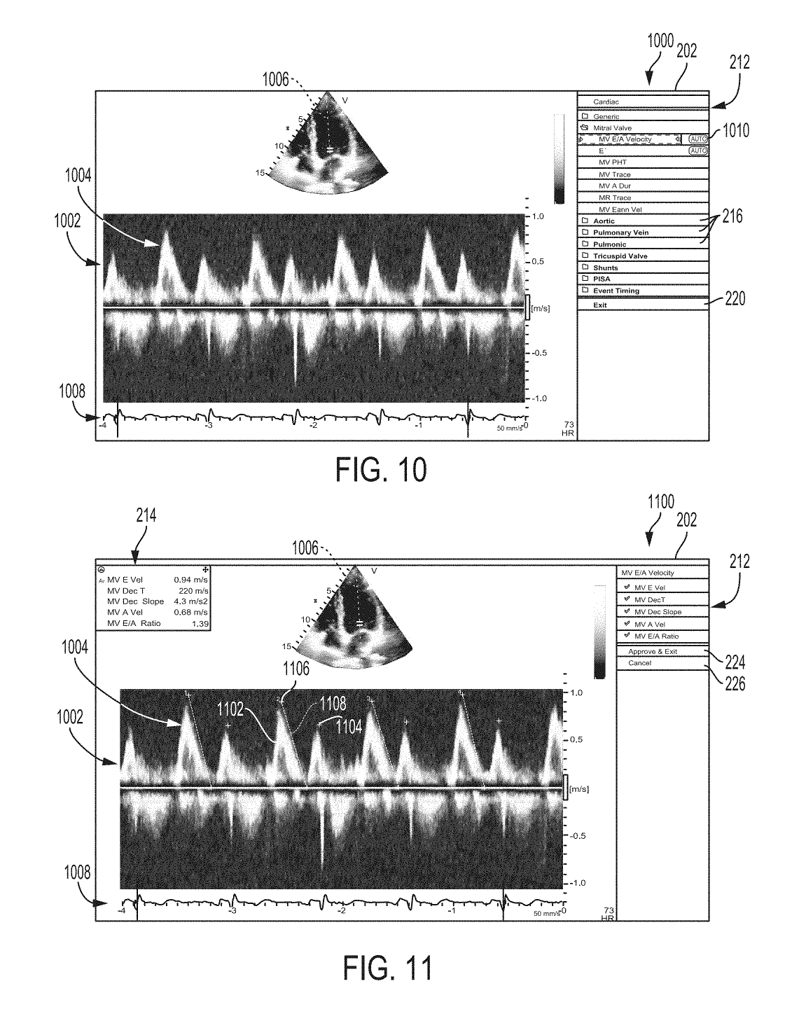

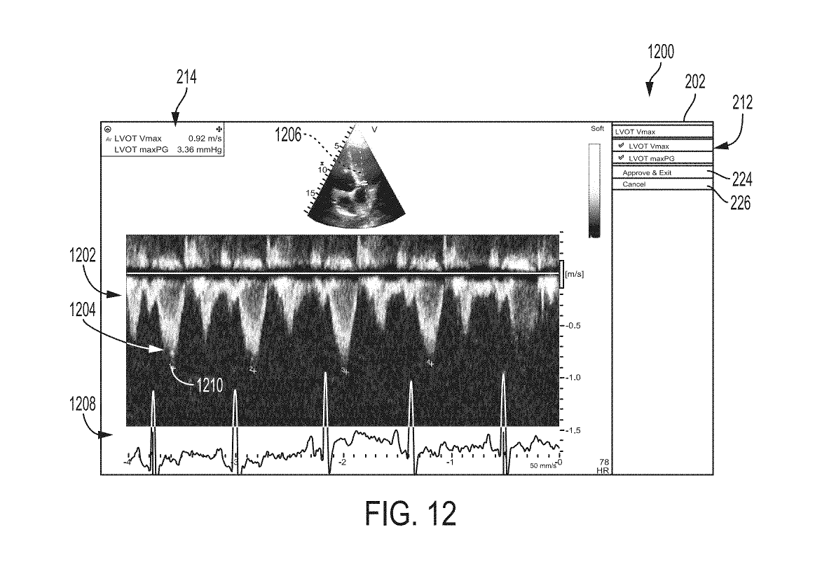

The following description relates to various embodiments of automatically analyzing a displayed spectrum containing multiple cycles (e.g., heart cycles) of data. The spectrum may be a Doppler spectrum generated from Doppler ultrasound data acquired with an ultrasound imaging system, such as the system shown in FIG. 1. Acquired Doppler data may then be used to generate the Doppler spectrum and display the Doppler spectrum via a display device of a user interface, such as the displayed Doppler spectrum of FIG. 2. FIGS. 2-6 and 10-12 show example display outputs of a display device of a user interface. For example, each of FIGS. 2-6 and 10-12 show a displayed Doppler spectrum and a menu display. Upon receiving a selection of a request to automatically determine one or more of a velocity boundary trace, a velocity point measurement, an E/A velocity measurement, or the like, a boundary trace for all cycles of the displayed Doppler spectrum may be automatically determined and then processed in order to display the corresponding measurement graphics (e.g., boundary trace line, E/A velocity measurement graphics including two points and a line for each cycle, or point measurement graphics including one or more points for each cycle). As one example, upon receiving a selection of a request to automatically trace a boundary of the envelope of each cycle of the displayed Doppler spectrum, a trace line is automatically determined and displayed on top of the Doppler spectrum, as shown in FIG. 3. The trace line may be updated in response to a user changing the desired tracing sensitivity level via a touch screen display, as shown in FIG. 8. A user may also individually review and analyze one or more individually traced cycles of the displayed Doppler spectrum, as shown in FIG. 4. One or more cycles may be excluded from the tracing, as shown in FIG. 5. After approving all remaining cycles of the traced Doppler spectrum, final measurement parameters may be displayed on the same display as the displayed Doppler spectrum, as shown in FIG. 6, and/or a worksheet may be generated and displayed showing the final measurement parameters for all approved cycles and for each individual cycle, as shown in FIG. 7. FIGS. 10-11 show examples of the E/A velocity measurements and displayed measurement graphics while FIG. 12 shows an example of a velocity point measurement and displayed measurement graphics. FIGS. 9A-9B show a method for automatically analyzing the displayed Doppler spectrum (e.g., displayed in any of FIGS. 2-6 and 10-12). In this way, a displayed Doppler spectrum may be automatically traced, analyzed, and have corresponding measurement graphics displayed on top of, thereby reducing analysis time and difficulty and therefore increasing an accuracy of diagnoses based on the analyzed Doppler spectrum.

FIG. 1 illustrates a block diagram of a system 100 according to one embodiment. In the illustrated embodiment, the system 100 is an imaging system and, more specifically, an ultrasound imaging system. However, it is understood that embodiments set forth herein may be implemented using other types of medical imaging modalities (e.g., MR, CT, PET/CT, SPECT etc.). Furthermore, it is understood that other embodiments do not actively acquire medical images. Instead, embodiments may retrieve image or ultrasound data that was previously acquired by an imaging system and analyze the image data as set forth herein. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of ultrasound systems, the system 100 may be a unitary system that is capable of being moved (e.g., portably) from room to room. For example, the system 100 may include wheels or be transported on a cart.

In the illustrated embodiment, the system 100 includes a transmit beamformer 101 and transmitter 102 that drives an array of elements 104, for example, piezoelectric crystals, within a diagnostic ultrasound probe 106 (or transducer) to emit ultrasonic signals (e.g., continuous or pulsed) into a body or volume (not shown) of a subject. The elements 104 and the probe 106 may have a variety of geometries. The ultrasonic signals are back-scattered from structures in the body, for example, blood vessels and surrounding tissue, to produce echoes that return to the elements 104. The echoes are received by a receiver 108. The received echoes are provided to a receive beamformer 110 that performs beamforming and outputs an RF signal. The RF signal is then provided to an RF processor 112 that processes the RF signal. Alternatively, the RF processor 112 may include a complex demodulator (not shown) that demodulates the RF signal to form IQ data pairs representative of the echo signals. The RF or IQ signal data may then be provided directly to a memory 114 for storage (for example, temporary storage). The system 100 also includes a system controller 116 that may be part of a single processing unit (e.g., processor) or distributed across multiple processing units. The system controller 116 is configured to control operation of the system 100.

For example, the system controller 116 may include an image-processing module that receives image data (e.g., ultrasound signals in the form of RF signal data or IQ data pairs) and processes image data. For example, the image-processing module may process the ultrasound signals to generate slices or frames of ultrasound information (e.g., ultrasound images) or ultrasound waveforms (e.g., continuous or pulse wave Doppler spectrum or waveforms) for displaying to the operator. When the system 100 is an ultrasound system, the image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. In one example, the system 100 may be an ultrasound system operating in a spectral Doppler mode and used to obtain a pulse wave Doppler spectrum or continuous wave Doppler spectrum (referred to herein as a Doppler spectrum) that includes blood flow velocity data for multiple heart cycles (e.g., beats). Examples of a Doppler spectrum displayed via a user interface of the ultrasound imaging system are shown in FIGS. 2-6 and 10-12, as described further below.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices or waveforms of acquired ultrasound information that are not scheduled to be displayed immediately. The image memory 120 may comprise any known data storage medium, for example, a permanent storage medium, removable storage medium, and the like. Additionally, the image memory 120 may be a non-transitory storage medium.

In operation, an ultrasound system may acquire data, for example, spectral Doppler data sets and/or volumetric data sets by various techniques (for example, 3D scanning, real-time 3D imaging, volume scanning, 2D scanning with probes having positioning sensors, freehand scanning using a voxel correlation technique, scanning using 2D or matrix array probes, and the like). Ultrasound spectrum (e.g., waveforms) and/or images may be generated from the acquired data (at the controller 116) and displayed to the operator or user on the display device 118.

The system controller 116 is operably connected to a user interface 122 that enables an operator to control at least some of the operations of the system 100. The user interface 122 may include hardware, firmware, software, or a combination thereof that enables an individual (e.g., an operator) to directly or indirectly control operation of the system 100 and the various components thereof. As shown, the user interface 122 includes a display device 118 having a display area 117. In some embodiments, the user interface 122 may also include one or more user interface input devices 115, such as a physical keyboard, mouse, and/or touchpad. In one embodiment, a touchpad may be configured to the system controller 116 and display area 117, such that when a user moves a finger/glove/stylus across the face of the touchpad, a cursor atop the ultrasound image or Doppler spectrum on the display device 117 moves in a corresponding manner.

In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as an input device that is configured to receive inputs from the operator. The display device 118 also communicates information from the controller 116 to the operator by displaying the information to the operator. The display device 118 and/or the user interface 122 may also communicative audibly. The display device 118 is configured to present information to the operator during or after the imaging or data acquiring session. The information presented may include ultrasound images, Doppler spectrum, graphical elements, measurement graphics of the displayed Doppler spectrum, measurement parameters of the displayed Doppler spectrum, user-selectable elements, and other information (e.g., administrative information, personal information of the patient, and the like).

In addition to the image-processing module, the system controller 116 may also include one or more of a graphics module, an initialization module, a tracking module, and an analysis module. The image-processing module, the graphics module, the initialization module, the tracking module, and/or the analysis module may coordinate with one another to present information to the operator during and/or after the imaging session. For example, the image-processing module may be configured to display an acquired image or Doppler spectrum on the display device 118, and the graphics module may be configured to display designated graphics along with the Doppler spectrum, such as selectable icons and measurement parameters (e.g., data) relating to the Doppler spectrum. The controller may include algorithms stored within a memory of the controller for analyzing an acquired Doppler spectrum and determining an outer boundary (e.g., tracing) of the Doppler spectrum, as well as measurement graphics and parameters of the Doppler spectrum, as described further below with reference to FIGS. 2-12.

The screen of the display area 117 of the display device 118 is made up of a series of pixels which display the data acquired with the probe 106. The acquired data includes one or more imaging parameters calculated for each pixel, or group of pixels (for example, a group of pixels assigned the same parameter value), of the display, where the one or more calculated image parameters includes one or more of an intensity, velocity (e.g., blood flow velocity), color flow velocity, texture, graininess, contractility, deformation, and rate of deformation value. The series of pixels then make up the displayed image and/or Doppler spectrum generated from the acquired ultrasound data.

As introduced above, the system 100 may be an ultrasound system operating in a spectral Doppler mode and used to obtain a Doppler spectrum (e.g., pulse wave or continuous wave Doppler) that includes blood flow velocity data. The acquired Doppler data may be used to generate the Doppler spectrum which may then be displayed via the display device 118 of the user interface 122. The displayed Doppler spectrum may include a blood flow velocity waveform signal over multiple heart cycles (e.g., beats). Examples of a Doppler spectrum displayed via a user interface of the ultrasound imaging system are shown in FIGS. 2-6 and 10-12, as described further below.

As introduced above, in order to accurately diagnose a patient's heart rhythm, multiple heart cycles of Doppler ultrasound data must be obtained (e.g., via an ultrasound imaging system, such as system 100 shown in FIG. 1) and analyzed (e.g., 3-5 cycles for patients in sinus rhythm and 5-10 cycles for patients with irregular heart rhythms). Data from the acquired cycles (which may include various blood flow measurement parameters such as maximum velocity, mean velocity, pressure gradient, and the like) may then be individually analyzed and averaged in order to accurately diagnose a patient. When three or more heart cycles are analyzed, medical technicians (e.g., echo specialists) may have to obtain both the average measurement parameters for all the acquired heart cycles and the detailed measurement parameters for each heart cycle of all the acquired heart cycles in order to decide whether to include or exclude the measurement parameters of a particular heart cycle. For example, in many cases it can be technically challenging to acquire robust or consistent data along the heart cycles of a pulse wave or continuous wave Doppler spectrum when analyzing Tricuspid Valve Regurgitation. In this example, an echo specialist may want to select only one heart cycle which contains the most representative continuous Doppler data. However, it may be time consuming and technically challenging to analyze the waveform of each heart cycle and then determine which cycles to include or exclude for the final analysis.

Further, as explained further below, in order to analyze the Doppler spectrum and obtain the measurement parameters, the Doppler spectrum displayed via the user interface must be traced (e.g., an outline or boundary of the Doppler waveform, forming a boundary between the blood flow velocity signal data and regions with no blood flow velocity signal data must be drawn). The measurement parameters are then determined, by the controller, for the traced areas of the Doppler spectrum. However, doing this manually (e.g., via an input device such as a mouse, stylist, or fingertip on a touch screen) for multiple heart cycles of data may be time consuming and may also have reduced accuracy.

FIGS. 2-8 and 10-12 show example display outputs of a user interface display (e.g., display device 118 of ultrasound imaging system 100 shown in FIG. 1). Specifically, FIGS. 2-6 and 10-12 show example display outputs showing a Doppler spectrum including blood flow velocity data over several heart cycles. FIG. 7 shows a report (e.g., worksheet) showing average and individual measurement parameters for a traced Doppler spectrum after a selected number of traced heart cycles have been accepted. FIG. 8 shows a touch panel display including a sensitivity input for adjusting a sensitivity of an automatic tracing of the Doppler spectrum.

Each of FIGS. 2-6 and 10-12 show an example display output displayed via a display device 202 of a user interface. As explained above with reference to FIG. 1, the display device may be configured to display waveforms, Doppler spectrum, and/or additional Doppler plots generated from ultrasound data acquired with an ultrasound imaging system (such as ultrasound imaging system 100 shown in FIG. 1). As shown in each of FIGS. 2-6 and 10-12, the display device 202 displays a first Doppler spectrum (e.g., a continuous or pulse wave Doppler spectrum) 204 (FIGS. 2-6), a second Doppler spectrum 1002 (FIGS. 10-11), or a third Doppler spectrum 1202 (FIG. 12), each of which includes blood flow signal data for several cycles (e.g., heart beats). As shown in FIGS. 2-6 and 10-12, the Doppler spectrum 204 is a continuous wave Doppler spectrum and Doppler spectrums 1002 and 1202 are pulsed wave Doppler spectrums; however, in alternate embodiments the displayed Doppler spectrum may be either a continuous or pulse wave Doppler spectrum. As seen in FIGS. 2-6 and 10-12, the Doppler spectrum have units of m/s and are plotted along an x-axis for several cycles (e.g., heart beats). The signal of the Doppler spectrum 204 has boundaries 206, the Doppler spectrum 1002 has boundaries 1004, and the Doppler spectrum 1202 has boundaries 1204, all of which follow along the positive and negative peaks of each cycle. Also in each of FIGS. 2-6 and 10-12, the display device 202 displays a 2D image 208 (FIGS. 2-6), 2D image 1006 (FIG. 10-11), or 2D image 1206 (FIG. 12) of the position of the continuous (or pulse) wave Doppler spectrum and an echocardiogram (ECG) plot 210 (FIGS. 2-6), 1008 (FIG. 10-11), or 1208 (FIG. 12) which display the waveform for each heart beat (e.g., cycle). For example, for continuous wave Doppler, the 2D image 208 includes a dotted line (e.g., line of Doppler) representing where the blood flow velocities shown in the Doppler spectrum 204 are measured. As such, if the position of this dotted line changes, the blood flow velocities on the Doppler spectrum 204 will also change. Alternatively, for pulsed wave Doppler, the Doppler spectrum will only show signals from a small depth range along the dotted line. Adjusting the depth range will hence change the blood flow velocities on the Doppler spectrum. The 2D image 208 is a color Doppler image where the colored portions represent blood flow (yellow colors indicate blood flow toward the ultrasound probe used to acquire the ultrasound data and blue color indicate blood flow away from the ultrasound probe). A color scale 209 for the 2D image 208 is also shown in FIGS. 2-6 (and a similar scale is shown in FIGS. 10-12).

Display device 202 also displays a menu 212 in each of FIGS. 2-6 and 10-12 and a measurement parameter display 214 in each of FIGS. 3-6 and 11-12. As shown in FIGS. 3-6 and 11-12, the menu 212, measurement parameter display 214, and the Doppler spectrum 204, 1002, or 1202 are all displayed at the same time on the same display device 202. The menu 212 includes different menu options including selectable measurement folder icons (e.g., buttons) 216 for accessing different folders (e.g., Aortic, Pulmonary vein, Pulmonic, and the like) of measurements that are applicable for different Doppler spectrum types including aortic, Mitral valve spectrums, Pulmonary vein spectrums, and the like.

As shown in FIG. 2, the AV Trace (boundary trace of the Doppler spectrum) icon is selected, an automatic trace icon (e.g., “AV Trace Auto” shown in FIG. 2) 218 located under a selected icon 216 (e.g., “Aortic” shown in FIG. 2), and exit icon 220, as shown in FIGS. 2 and 6. After selecting the AV Trace icon (for the automatic multicycle Doppler spectrum measurements), the menu 212 may also display various measurement parameter icons 222 which may be selected (as seen by a check mark next to the selected measurement parameter icon) and deselected via a user interface input device (e.g., via a user), as well as an approve and exit icon 224, and a cancel icon 226, as shown in FIGS. 3-5. A selection of the cancel icon 226 may result in abortion of the Doppler spectrum measurement, at any time in the workflow, and discarding of all measurement parameter results. This function may be used if the automatic algorithm failed to detect any trace/points, or if the user is not satisfied with the detected trace/points, and decides to do manual measurements instead. The selected measurement parameters, selected via the various measurement parameter icons 222, are then determined (e.g., automatically calculated) for the traced Doppler spectrum (as explained further below) and displayed via the measurement parameter display 214.

Turning to FIG. 2, an example display output 200 of display device 202 showing the Doppler spectrum 204 is presented. The display output 200 shown in FIG. 2 may be a display output displayed after selecting the Aortic and AV Trace icons 216. For example, under the Aorta icon 216, the “AV Trace” icon is selected (as seen by the check mark). However, the automatic trace icon 218 has not yet been selected and thus the Doppler spectrum 204 is displayed without tracing the boundary 206.

After the automatic trace icon 218 is selected (e.g., via a user interface input device, such as a mouse, keyboard, or touchscreen icon selectable via a user's finger or stylist), a processor or controller in communication with the display device 202 (such as processor 116 shown in FIG. 1) may calculate an automatic tracing of the boundary 206 of the Doppler spectrum 204 using one or more algorithms stored in a memory of the processor, where the Doppler spectrum boundary data and user inputs, such as a tracing sensitivity, are inputs into the one or more algorithms. In one example, the algorithm for automatically tracing the boundary 206 may be additionally based on one or more of a noise level estimation, smoothing and thresholding, and/or combined with ECG trig points and a priori knowledge of blood flow patterns for Doppler spectrums acquired at specific anatomical positions.

FIG. 3 shows an example display output 300 of the display device 202 showing the automatic tracing applied to the Doppler spectrum 204, which is displayed as a result of receiving a signal that the automatic trace icon 218 was selected. Specifically, a trace line 302 is applied to the boundary 206 of the Doppler spectrum 204, automatically without additional input from a user (other than selecting the automatic trace icon 218 shown in FIG. 2). In this way, the processor may automatically apply the trace line 302 (also referred to as a boundary line or an outline) to the boundary 206 of the signal area of the displayed Doppler spectrum 204 without a user identifying or selecting any of the points of the boundary 206 for tracing. This may reduce user error and variation in applying the trace line 302, as well as reducing the time to trace multiple heart cycles of Doppler data (four cycles shown in FIG. 3). More specifically, the trace line 302 is applied to the envelope 303 (e.g., negative peaks) of each heart cycle. In alternate embodiments, such as in the mitral valve E/A velocity measurements described below, the trace line may be applied to the positive peaks in addition to or instead of the negative peaks.

The trace line 302 and the area within the trace line 302 may include blood flow velocity data of the Doppler spectrum (e.g., a signal area) while the area outside of the trace line 302 may not include usable blood flow velocity data (or a strong enough signal). In this way, automatically tracing the Doppler spectrum may include drawing the trace line 302 on top of the displayed Doppler spectrum 204 between where there is signal and isn't signal, as a continuous line for each envelope (for each cycle) of the Doppler spectrum 204. The placement of the trace (e.g., drawn boundary) line 302 may be adjusted (e.g., moved closer inward or outward relative to the signal area of the Doppler spectrum 204) based on a set sensitivity level. For example, each pixel of the display area of the Doppler spectrum 204 of the display device 202 may have a blood flow velocity value and an associated display color (e.g., black where there is no signal and yellow where there is signal). However, some pixels along the boundary 206 may appear fuzzier and have varying values. The sensitivity of the tracing may determine which of these pixels to include and exclude from the boundary (and within the boundary) of the trace line 302. In some embodiments, smoothing of both the Doppler spectrum data and the trace line 302 may be performed due to noisiness of the Doppler spectrum data. While automatic tracing of the Doppler spectrum 204 is described above, if a user does not select the automatic trace icon 218, a user may manually measure and place the trace line (e.g., without using the automatic algorithm of the system).

The sensitivity level of the automatic tracing may be set and adjusted via a user through a selectable display of the user interface. FIG. 8 shows an example of a selectable display, in the form of a touch panel (e.g., screen) display 800, including a sensitivity input 802 for adjusting the sensitivity of the automatic tracing of the Doppler spectrum. In one embodiment, the touch panel display 800 may be a separate touch-sensitive display 801 than the display device 202, but selections via the touch panel display 800 may influence what is displayed via the display device 202. In this embodiment, both the display device 202 and the touch panel display 800 may be part of the same user interface. In an alternate embodiment, the user interface may not include a separate touch panel display and instead the display device 202 may include soft-buttons on the screen to perform many of the actions described herein with regard to the touch panel display 800. The touch panel display 800 may be a touch sensitive display that receives user inputs via user touching the display 801 and/or via a plurality of rotary buttons (e.g., a user interface input device of the user interface) that coincide with the rotary inputs 803 shown on display 801 (which include sensitivity input 802). For example, the rotary inputs 803 may show what each of the rotary buttons adjust (e.g., show the positions of and corresponding adjustments of the rotary buttons). The sensitivity input may be a rotary button that is adjustable via a user and that results in adjustment of the sensitivity level of the automatic tracing (e.g., the placement of trace line 302 shown in FIGS. 3-6). The touch panel display 800 also includes a worksheet input (e.g., button) 804 that may display a worksheet (e.g., worksheet 700 shown in FIG. 7, as described further below), a cancel input 808 that may cancel the current automatic Doppler measurement workflow (as described herein with reference to FIGS. 2-6), an approve and exit input 810 that may approve the current selection and then return to the previously displayed display screen, and a recalc input 812 that may recalculate (e.g., rerun) the automatic detection (e.g., tracing or other measurement parameters), and a horizon sweep input 805 that may scale the Doppler spectrum along the x-axis (time axis).

As shown in FIG. 3, four different heart cycles of the Doppler spectrum 204 are automatically traced. The blood flow velocity data of the Doppler spectrum 204, along the applied trace line 302, is then used by the processor to determine (e.g., estimate or calculate) the selected measurement parameters (e.g., the measurement parameters selected via the measurement parameter icons 222 of the menu 212). The determined measurement parameters are then displayed via measurement parameter display 214. The determined measurement parameters are average measurement parameters which are averaged over all the traced cycles of the Doppler spectrum 204 (e.g., averaged over all four traced cycles shown in FIG. 3), as indicated by the average icon (“Av”) 304 shown at the top left of the measurement parameter display 214. As an example, and as shown in FIG. 3, the displayed average measurement parameters include average maximum blood flow velocity (AV Vmax), an average mean blood flow velocity (AV Vmean), an average maximum pressure gradient of the blood flow velocity (AV maxPG), an average mean pressure gradient of the blood flow velocity (AV meanPG), average velocity time integral (AV VTI), and average envelope time (AV Env. Ti, which may be referred to as the ejection time). While these six measurement parameters are shown in display output 300, in alternate embodiments, more, less, and/or different measurement parameters may be calculated and then displayed for the traced Doppler spectrum. During a display session, a user may select additional or deselect some of the measurement parameter icons 222. As a result, the processor may automatically determine any newly selected measurement parameters and update the measurement parameters displayed via the measurement parameter display 214.

As shown by the example display output 400 of FIG. 4, during a display session, a user may select, via a user interface input device, one cycle out of all of the traced cycles of the Doppler spectrum 204. For example, a user may positon a mouse, a finger, or a stylist over a display area of one of the traced cycles (e.g., the third cycle 402, as shown in FIG. 4). In another example, the user may manually select a cycle area 404 of the third cycle 402 along the x-axis of the Doppler spectrum plot via the user interface input device. In response to receiving the selection of the individual cycle (e.g., third cycle 402), the processor calculates and then displays the measurement parameters for only the selected third cycle 402. In this way, the measurement parameter display 214 is updated to display the selected measurement parameters for only the third cycle 402 and no other cycle, as indicated by the cycle icon 406 (e.g., “3”) shown at the top left of the measurement parameter display 214. Thus, the processor uses only the blood flow velocity data along the trace line 302 of the third cycle 402 to determine the measurement parameters displayed via the measurement parameter display 214 in FIG. 4. When none of the individual cycles are being selected, the measurement parameter display 214 displays the measurement parameters averaged over all cycles of the traced Doppler spectrum 204.

A user may focus on the different individual traced cycles of the Doppler spectrum 204 (via moving the user interface input device around the screen, over the displayed Doppler spectrum 204, or by manually selecting one of the cycles) in order to see the measurement parameters for the currently selected cycle. As a result, a user may more easily and quickly determine which cycles of the traced Doppler spectrum 204 should be kept for final analysis (and for final patient diagnosis) and which should be discarded. For example, some cycles may be outliers and may not be representative of a patient's heart condition (and thus should be discarded). In another example, one cycle out a plurality of cycles may show an abnormality that should be analyzed alone (and thus the other, remaining cycles should be discarded). By automatically calculating and displaying the individual cycle measurement parameters, a user may more quickly and accurately determine which data from which cycles of the traced Doppler spectrum should be kept and used to diagnose a patient.

While selecting an individual cycle, as shown in FIG. 4, an accept (or select) icon 408 and a reject (e.g., discard or deselect) icon 410 are show within a display area of the selected cycle. A user may select one of these two icons using the user interface input device. In response to receiving a selection of the accept icon 408, the processor may discard all other cycles of the Doppler spectrum, except for the currently selected individual cycle. In response to receiving a selection of the reject icon 410 (e.g., discard or deselect input), the processor may discard the traced data for the selected cycle. As a result, the display device 202 may update the automatic tracing to only apply the trace line 302 to the cycles of the Doppler spectrum 204 that have not be deselected (e.g., the selected cycles), as shown in FIG. 5. Specifically, FIG. 5 shows an example display output 500 where the trace line 302 is applied to the first, second, and fourth cycle of the Doppler spectrum 204, but not the rejected third cycle 402. The measurement parameter display 214 is updated to display the average measurement parameters averaged over only the selected traced cycles (e.g., the remaining first, second, and fourth cycles, in the example of FIG. 5). Thus, the measurement parameter display 214 of FIG. 5 shows the average measurement parameters averaged over cycles one, two, and four while the measurement parameter display 214 of FIG. 3 shows the average measurement parameters averaged over cycles one, two, three, and four. In this way, after a user is finished selecting/rejecting an individual cycle, the measurement parameter display returns to displaying the measurement parameters averaged over all the selected (e.g., not rejected) traced cycles. Once a user is satisfied with the remaining traced cycles of the Doppler spectrum 204, they may select the approve and exit icon 224.

In response to receiving a user input approving all the remaining traced cycles (e.g., a signal that the approve and exit icon 224 has been selected or the corresponding approve and exit button 810 on the touch panel display 800 has been selected), the display device 202 displays the finally selected traced cycles and measurement parameters for each finally selected traced cycle, as shown in FIG. 6. Specifically, FIG. 6 shows an example display output 600 showing the trace outline 302 on the three finally selected cycles of the Doppler spectrum 204 (e.g., cycles one, two, and four). In response to approving the remaining cycles, the measurement parameter display 214 is updated to display the measurement parameters for each individual cycle of the finally selected cycles (e.g., individual measurement parameters for cycles one, two, and four, shown as the finally selected cycles one, two, and three in the measurement parameter display 214 in FIG. 6). After approving the remaining cycles, the measurement parameters of the approved cycles of the traced Doppler spectrum may be stored in a memory of the processor coupled with the user interface.

FIG. 7 shows an example display output 700 of the user interface showing a worksheet (e.g., report) 702 generated for the approved (and stored) measurement parameters of the approved cycles of the Doppler spectrum of FIG. 6. The worksheet 702 may be generated in response to receiving an approval of the finally selected cycles and/or a selection of a worksheet input, such as a dedicated physical button on the front panel of the ultrasound system for entering/exiting the worksheet, or the worksheet input 804 on the touch panel display 800. As shown in FIG. 7, the worksheet includes a measurement parameter column 704 listing the desired (e.g., user selected) measurement parameters, an average value column 706 that lists the values of the listed measurement parameters, averaged over all the approved cycles of the Doppler spectrum, and an individual value column 708 that lists the values of the listed measurement parameters for each individual cycles for the approved cycles. As shown in FIG. 7, the worksheet 702 includes three individual value columns 708 since the final approved Doppler spectrum from FIG. 6 included data from three heart cycles. However, in alternate embodiments, the worksheet may include more or less than three individual value columns 708 based on the number of approved cycles in the Doppler spectrum. In this way, a user may view, via one worksheet and screen, both the average measurement parameters for all approved cycles and measurement parameters for each approved cycle. The worksheet may also gather all the measurement parameters for an entire ultrasound exam, which may be measured on several different images (e.g., images in addition to the Doppler spectrum shown in FIGS. 2-6).

In alternate embodiments, additional automatic Doppler measurements may be performed without displaying a boundary trace (e.g., trace line 302). For example, FIGS. 10-11 show example display outputs for an automatic mitral valve E/A velocity measurement and FIG. 12 shows and example display output for an automatic velocity point measurement. Both of these examples may follow a similar procedure as described above with reference to FIGS. 2-6 and similar sensitivity controls and worksheet outputs to those shown in FIGS. 7-8 may be used or generated for these embodiments.

Specifically, FIG. 10 shows an example display output 1000 of the display device 202 for an automatic mitral valve E/A velocity measurement. The display device 202 displays a Doppler spectrum 1002 having a boundary 1004, an ECG plot 1008, a 2D image 1006, and menu 212. The acquired Doppler spectrum that is displayed may be a blood flow velocity spectrum of a mitral valve. With the Doppler spectrum 1002 displayed, a user may select an automatic measurement icon 1010 from the menu 212 in order to activate the automatic E/A velocity measurement. The mitral valve E/A velocity measurement may include automatically performing the boundary trace detection (e.g., tracing the boundary of the displayed Doppler spectrum for multiple heart cycles, similarly to as described above with reference to FIG. 3) in the background, and then analyzing the boundary trace to detect three features. The three features include: a maximum E wave velocity, a deceleration slope, and a maximum A wave velocity. When the mitral valve opens during diastole, blood flows in from the left atrium to the left ventricle. This is called the early filling phase, and the flow in this phase is referred to as the E wave. The maximum velocity of the E wave is the first point (labeled as 1106 in FIG. 11, as described further below) in the E/A velocity measurement. The right flank of the E wave signal shows how rapidly flow velocity declines in the early filling phase. The deceleration slope is a straight line that is fit to the right flank of the flow boundary trace for the E wave. The displayed measurement parameters (the line labelled as 1108 in FIG. 11, as described further below) from this line are the slope and the deceleration time (the duration from maximum E velocity until the deceleration line reaches zero velocity). At the end of diastole, the left atrium contracts, forcing more blood into the left ventricle. This gives a second inflow wave in the Doppler spectrum, known as the A wave. The maximum velocity of the A wave is the second point in the E/A velocity measurement (labeled as 1104 in FIG. 11, as described further below).

As shown in the example display output 1100 of FIG. 11, in response to a user selecting the automatic measurement icon 1010 and after the boundary trace (e.g., trace line) of the displayed Doppler spectrum 1002 is determined by the processor, the E/A velocity measurement graphics are displayed (e.g., marked) on top of the Doppler spectrum 1002. The E/A velocity measurement graphics that are displayed include the E wave velocity point 1106, deceleration slope line 1108, and the A wave velocity point 1104. As shown in FIG. 11, these measurement graphics are automatically marked and displayed (e.g., without user input other than selecting the automatic measurement icon 1010) along each positive peak 1102 for each cycle of the Doppler spectrum 1002. In this way, two points and a line our labeled automatically on top of the displayed Doppler spectrum 1002 for each cycle of the Doppler spectrum 1002 and the position of these measurement graphics is determined automatically by the processor. As seen in FIG. 11, measurement parameters for the E/A velocity measurement (which correspond to the displayed measurement graphics) are automatically displayed via the measurement display 214. These may be displayed as average parameter values for all cycles of the Doppler spectrum 1002 until a single cycle of the spectrum is selected, similarly to as explained above with reference to FIG. 4. As explained above with reference to FIGS. 4-5, a user may reject any of the cycles or select a single cycle, thereby rejecting the remaining cycles. This would result in updating of the displayed measurement parameters and the measurement graphics would only be displayed for the non-rejected (or selected) cycles).

FIG. 12 shows an example display output 1200 of the display device 202 for an automatic velocity point measurement. The display device 202 displays a Doppler spectrum 1202 having a boundary 1204, an ECG plot 1208, a 2D image 1206, and a menu 212. A user may select an automatic measurement icon (in this example, the automatic measurement icon shown in FIG. 2 next to “LVOT Vmax) from the menu 212 in order to activate the automatic maximum velocity point measurement for the left ventricular outflow tract (LVOT). Though FIG. 12 and the description below describes the automatic velocity point measurement for the LVOT, in alternate embodiments, a similar point measurement may be performed for alternate point measurements (where one or more points are displayed on each cycle of the Doppler spectrum) of the Doppler spectrum, such as an AV Vmax (maximum velocity for atrial valve), TR Vmax (maximum velocity for tricuspid valve), an E′ measurement (a point at the maximum velocity of the wall of the heart by the mitral valve during E wave), or the like.

After a user selects the automatic measurement icon for the velocity point measurement, the processor may automatically perform the boundary trace detection (e.g., tracing the boundary of the displayed Doppler spectrum for multiple heart cycles, similarly to as described above with reference to FIG. 3) in the background, and then analyze the boundary trace to detect the selected velocity point (e.g., the point with the maximum velocity for each cycle of the Doppler spectrum). The display output 1200 of FIG. 12 shows the detected maximum velocity points 1210 for each cycle of the Doppler spectrum 1204. As shown in FIG. 12, the boundary trace itself is not displayed via display device 202, only the velocity points 1210. All measurement parameters determined from the automatic trace and that relate to the single points 1210 are automatically displayed (e.g., maximum velocity and maximum pressure gradient) via measurement parameter display 214. These measurement parameters may be displayed as average parameter values for all cycles of the Doppler spectrum 1202 until a single cycle of the spectrum is selected, similarly to as explained above with reference to FIG. 4. As explained above with reference to FIGS. 4-5, a user may reject any of the cycles or select a single cycle, thereby rejecting the remaining cycles. This would result in updating of the displayed measurement parameters and the measurement graphics would only be displayed for the non-rejected (or selected) cycles).

In still other embodiments, two-point measurements (instead of just the single point measurement shown in FIG. 12) may be automatically determined based on an automatically determined boundary trace of the Doppler spectrum. For a two-point measurement, the following measurement parameters may be derived: the velocity at both points, the difference between the two velocities, the ratio between the two velocities, the time difference between the two points, and/or the slope (acceleration/deceleration) of the line between the two points.

FIGS. 9A-9B show a flow chart of a method 900 for automatically analyzing a displayed Doppler spectrum containing multiple heart cycles of data. The displayed Doppler spectrum may be displayed via a display device of a user interface of an ultrasound system (such as display device 118 shown in FIG. 1 and display device 202 shown in FIGS. 2-6). The displayed Doppler spectrum may be similar to the Doppler spectrum 204 shown in FIGS. 2-6, the Doppler spectrum 1002 shown in FIGS. 10-11, or the Doppler spectrum 1202 shown in FIG. 12, as described above. Instructions for carrying out method 900 may be executed by a controller or processor (such as processor 116 shown in FIG. 1) based on instructions stored on a memory of (or coupled to) the processor and in conjunction with signals received from user interface input devices (such as a mouse, keyboard, or touch-sensitive display) and an ultrasound probe in communication with the processor, such as the user interface input devices and probe described above with reference to FIG. 1. The processor may update the outputs of the display device (e.g., what measurement parameters, data, and images are displayed on the display device), according to the methods described below.

The method begins at 902 by displaying an acquired Doppler spectrum (e.g., a Doppler spectrum generated from acquired Doppler ultrasound data) via a user interface (such as user interface 122 shown in FIG. 1) and displaying a user interface display for selecting an auto trace input. As one example, the user interface may be a display device, such as display device 202 shown in FIGS. 2-6 and 10-12. Displaying the Doppler spectrum may include displaying a continuous or pulse wave Doppler spectrum, such as Doppler spectrum 204 shown in FIG. 2-6, the Doppler spectrum 1002 shown in FIGS. 10-11, or the Doppler spectrum 1202 shown in FIG. 12, via the display device which may be viewable by a user. The method at 902 may also include displaying one or more of a 2D image of the position of the continuous (or pulse) wave Doppler spectrum (such as 2d image 208 shown in FIGS. 2-6, 1006 shown in FIGS. 10-11, and 1206 shown in FIG. 12), an echocardiogram (ECG) plot (such as ECG plot 210 shown in FIGS. 2-6, 1008 shown in FIGS. 10-12, and 1208 shown in FIG. 12), and a menu display (such as menu 212 shown in FIGS. 2-6 and 10-12). Displaying the menu via the display device may include displaying an automatic (e.g., auto) measurement icon (such as automatic trace icon 218 shown in FIG. 2, automatic LVOT Vmax measurement icon shown in FIG. 2, or automatic measurement icon 1010 shown in FIG. 10). As one example, the Doppler spectrum and other Doppler data/images may be displayed in response to the processor receiving a user input selecting ultrasound data from a thumbnail on a clipboard of the display device and/or during a real-time scanning session.

At 904, the method includes determining whether an auto measurement input signal has been received. As one example, when a user selects (via a user interface input device) one of the displayed automatic measurement icons (e.g., auto trace icon 218 shown in FIG. 2, automatic LVOT Vmax measurement icon shown in FIG. 2, or automatic measurement icon 1010 shown in FIG. 10), the processor may receive a signal indicating that automatic tracing of the boundary (including the envelopes of each heart beat) and corresponding automatic determination of measurement graphics and parameters has been requested. As explained above, the measurement graphics may include one or more of the trace line of the automatic tracing of the boundary (as shown in FIG. 3), one or more points on the Doppler spectrum (e.g., such as maximum velocity for each cycle, as shown in FIG. 12), or an E/A measurement graphic including two points and a deceleration slope line between the two points (as shown in FIG. 11). Each measurement graphic may include corresponding measurement parameters that may include one or more of an average maximum blood flow velocity, an average mean blood flow velocity, an average maximum pressure gradient of the blood flow velocity, an average mean pressure gradient of the blood flow velocity, average velocity time integral, and average envelope time, an average blood flow velocity, an average pressure gradient of the blood flow velocity, average acceleration or deceleration times of blood flow velocity, average acceleration or deceleration slopes of blood flow velocity, and/or average ratios between one or more additional measurement parameters.

In response to receiving the input signal at 904, the method continues to 906 to automatically trace the cycles (e.g., the envelope, or negative or positive peaks, of each heart beat or cycle) of the displayed Doppler spectrum and then display the chosen measurement graphics via the display device of the user interface. As described above, automatically tracing the Doppler spectrum may include determining a placement of a trace line (e.g., trace line 302 shown in FIG. 3) based on a boundary of the signal area of the envelopes of the Doppler spectrum and a set or default sensitivity level of the tracing. For example, the processor may determine the trace line (e.g., the positioning and alignment of the trace line along a boundary of the Doppler spectrum, the boundary defined as a boundary between where there is a blood flow velocity signal/data and where there is not blood flow velocity signal/data) through a determination that directly takes into account the blood flow velocity values of the displayed Doppler spectrum, at each pixel of the display, and a set or default sensitivity level of the tracing (e.g., where within the boundary area to place the trace line). As another example, the controller may make a logical determination of the placement of the trace line along a perimeter of the envelopes of the displayed Doppler spectrum based on logic rules that are a function of the blood flow velocity signals/data of the Doppler spectrum at each pixel proximate to the boundary between no signal data and signal data and the set sensitivity level. In one example, the processor may determine the boundary trace (e.g., trace line) of the Doppler spectrum in the background (e.g., in the processor) but not display the determined trace line. Instead, the trace line may be used to determine the measurement graphics and corresponding measurement parameters. In another example, the processor may generate a signal to send to the display device for displaying the determined trace (e.g., boundary or outline) line. Displaying the determined trace line may include displaying a continuous line, all at once and without continuous user input, along a boundary of each envelope of each heart cycle of the displayed Doppler spectrum. In this way, automatically tracing the cycles of the Doppler spectrum at 906 may include automatically determining and, optionally (when selected, such as when the auto trace input is received) displaying the trace line on top of the displayed Doppler spectrum (e.g., the trace line and Doppler spectrum are displayed at the same time on the same display device) in response to receiving the auto measurement input signal only and without receiving any additional input from a user (such as selecting any pixels or points proximate to the boundary of the Doppler spectrum with a user interface input device). Tracing the Doppler spectrum may also be referred to herein as outlining a boundary of the Doppler spectrum or drawing a boundary line of the Doppler spectrum. As described above, automatically tracing the cycles of the Doppler spectrum at 906 may additionally or alternatively include determining and displaying a set of points at detected landmarks that are determined from the automatically determined boundary trace of the Doppler spectrum, such as for a mitral valve E/A velocity measurement or maximum velocity point measurements. For example, when points, not traces, are displayed it is possible for the user do adjust the position of the detected points before approving. Either by adjusting sensitivity, which will affect all points, or by double-clicking a single point, moving it, and single-clicking to place it at a new position.

At 908, the method includes determining whether a tracing sensitivity input (e.g., updated input) has been received. In one example, the processor may receive an input via a user interface input device selecting or changing a sensitivity level of the automatic tracing. For example, as described above with reference to FIG. 8, a user may change the sensitivity level used to determine the boundary trace line for the Doppler spectrum via a sensitivity input 802 displayed via a touch panel display. The processor may receive a signal with the updated sensitivity level in response to the user adjusting a setting or position of the sensitivity input. If no new or adjusted sensitivity input is received, the method continues to 910 to maintain the current automatic tracing setting and maintain the currently determined (and displayed, in some embodiments) trace line on the displayed Doppler spectrum. However, if a new or updated tracing sensitivity input is received, the method continues to 912 to update the automatic tracing based on the received tracing sensitivity input (e.g., based on the updated sensitivity level). Updating the automatic tracing may include determining (e.g., calculating) an updated trace line for the displayed Doppler spectrum using the updated sensitivity level and then updating the display of the measurement graphics over the Doppler spectrum (e.g., updating the positioning of the trace line on the displayed Doppler spectrum). Each time a changed sensitivity input is received, updated measurement parameters may then be determined in response to the updated measurement graphics.

At 914, the method includes displaying measurement parameters for the displayed Doppler spectrum, averaged over all traced cycles (e.g., heart beats or cycles). Displaying the measurement parameters at 914 may include a processor determining (e.g., calculating) the selected measurement parameters (e.g., the measurement parameters selected via measurement parameter icons 222 of menu 212, as shown in FIGS. 3-5 and FIGS. 11-12) and then displaying the determined selected measurement parameters via the display device (e.g., via measurement parameter display 214 shown in FIGS. 3-6 and 11-12). The measurement parameter names and the values of each measurement parameter may be displayed via the measurement parameter display, at the same time as displaying the traced Doppler spectrum. As one example, calculating the selected measurement parameters may include the processor calculating the selected measurement parameters according to equations or relationships for each measurement parameter (stored in memory) which are a function of the blood flow velocity data along the determined trace line of the Doppler spectrum. The determined and displayed measurement parameters at 914 (and 920, as described further below) may be average measurement parameters that are averaged over all of the traced cycles of the Doppler spectrum.

At 916, the method includes determining whether an updated selection of the displayed measurement parameters has been received. For example, the processor may receiving a signal (e.g., user input) that a user has selected additional or deselected some of the previously selected measurement parameters via a user interface input device. In response to this received signal, the processor may update the displayed measurement parameters (e.g., displayed via the measurement parameter display) at 920. As one example, the processor may determine the additionally selected measurement parameters and update the measurement parameter display to display the original and newly selected (and determined) measurement parameters). As another example, the processor may update the measurement parameter display to remove any deselected measurement parameters from the measurement parameter display. In some embodiments, the method at 920 may also include updating the displayed measurement graphics in response to the received signal that the user has selected additional or deselected some of the previously selected measurement parameters. For example, in the mitral valve E/A velocity measurement case, three points (E wave velocity, deceleration slope end, A wave velocity) may be displayed. If the user deselects display of A Vel and E/A ratio, the A wave velocity point may be removed from the measurement graphics (e.g., from the displayed Doppler spectrum).

If no signal of an updated selection of measurement parameters is received at 916, the method instead continues to 918 to maintain the currently displayed measurement parameters on the measurement parameter display.

The method continues to 922 in FIG. 9B. At 922, the method includes determining whether an input selection of an individual cycle (e.g., individual envelope) of the Doppler spectrum (which includes data from multiple heart cycles) has been received. Receiving an input selection of an individual cycle may include one or more of receiving a signal indicating that a user interface input device is positioned over top of one of the individual cycles (e.g., a mouse is positioned over the third cycle of the displayed Doppler spectrum) or a signal indicating that a user has selected a section of the Doppler spectrum, along the x-axis, which corresponds to an individual cycle (e.g., a mouse or alternate user interface input device has manually selected the section of the Doppler spectrum corresponding to the third cycle of the displayed Doppler spectrum, as shown in the example shown in FIG. 4). If no signal indicating that an input selection of an individual cycle has been received, the method continues to 924 to maintain the currently displayed measurement parameters which are averaged over all the traced cycles of the displayed Doppler spectrum (and not an individual cycle). Alternatively, if the processor has received an input selection of an individual cycle, the method continues to 926 to display the selected measurement parameters for the selected individual cycle. For example, the method at 926 may include updating the measurement parameter display to display the same measurement parameters but update the values displayed for each measurement parameter to be averaged over only the selected individual cycle. Thus, the method at 926 may include calculating the measurement parameters for the selected individual cycle using blood flow velocity data along the trace line of only the selected individual cycle (e.g., use only data along the trace line for the third traced envelope of the Doppler spectrum).

The method continues from both 926 and 924 to 928 where the method includes determining whether an input selection of one or more cycles out of all cycles of the traced Doppler spectrum to include/exclude from the traced data has been received. As one example, the processor may receive a signal from the user interface indicating that a user has accepted (e.g., selected) or rejected (e.g., discarded) one or more cycles of the traced Doppler spectrum. For example, as shown in FIG. 4, upon selecting an individual cycle, an accept icon (e.g., accept icon 408) or reject icon (e.g., reject icon 410) may be displayed in response to receiving an input selection of an individual signal. A user may then either select the accept icon to keep only the selected individual cycle (and reject the other, remaining cycles) within the traced Doppler data or select the reject icon to discard the data from the selected individual cycle from the traced Doppler data. If no input selection of the cycles to include/exclude has been received, the method continues to 930 to maintain the currently displayed measurement parameters for all the traced cycles. Alternatively, if an input selection of one or more cycles to include/exclude has been received, the method continues to 932 to discard the data of the excluded (e.g., rejected) cycle(s) and then update (e.g., re-calculate) and display the measurement parameters for all the remaining cycles of the traced Doppler spectrum. For example, the displayed measurement parameters may be automatically updated using only a selected number of cycles out of the total number of cycles of the Doppler spectrum, where the selected number is less than the total. For example, the method at 932 may include re-calculating the measurement parameters for (e.g., averaged over) all the remaining traced cycles and then updating the measurement parameter display with the updated measurement parameters, as shown in the example of FIG. 5. The method at 932 may also include updating the displayed measurement graphics to only display the measurement graphics for the selected (e.g., non-rejected) cycles. For example, as shown in FIG. 5, discarding the data of the excluded cycle may also include updating the displayed trace line to only be shown on the remaining cycles and not on the rejected cycle.

The method at 934 includes determining whether an input approving the remaining cycles of the traced Doppler spectrum has be received. As one example, the processor may receive a signal indicating that the user has approved all the remaining traced cycles. For example, as shown in FIG. 5, a user may select an approve icon (e.g., approve and exit icon 224) displayed via the display device. If the processor has not received the user input approving the remaining cycles, the method continues to 936 to maintain the currently displayed measurement parameters for all cycles and wait for the approval input.

Alternatively at 934, in response to receiving the user input, via the user interface, approving the remaining traced cycles, the method continues to 938 to update the measurement parameter display to display the measurement parameters for each individual cycle of the approved (e.g., finally selected) cycles. (e.g., as shown FIG. 6). After approving the remaining cycles, the measurement parameters of the approved cycles of the traced Doppler spectrum may be stored in a memory of the processor coupled with the user interface.

At 940, the method may optionally include generating and displaying a worksheet (e.g., report) with the Doppler spectrum measurement parameters for each approved cycle and average data for all approved cycles of the Doppler spectrum in response to receiving an input requesting display of the worksheet. For example, in response to receiving an approval of all remaining cycles, the processor may calculate values (e.g., average values which are averaged over each envelope of each cycle) for the selected measurement parameters for each approved cycle (e.g., individual measurement parameters) and for all approved cycles (e.g., average over all approved cycles). The worksheet may also contain measurement parameters from additional images in the same exam where a same kind of analysis has been done. For example, if an automatic AV Trace analysis on two Doppler spectrums is performed, each yielding results from three cycles, the worksheet will contain six sets of AV Trace results, each shown as individual results, and with a common average in worksheet. An example of a worksheet that may be displayed is shown in FIG. 7, as described above.

At any time during method 900, the processor may receive a user input cancelling the automatic analysis workflow (e.g., via the cancel buttons 226 and/or 808, as described above). In response to receiving this cancel input, the current analysis and measurements of the Doppler spectrum will be aborted and all measurement parameter results and the trace of the Doppler spectrum may be discarded. A user may then chose to select a different Doppler spectrum for analysis or manually trace and measure the Doppler spectrum.

In this way, a Doppler spectrum including acquired Doppler data from a plurality (e.g., at least two, two or more, or three or more) of heart cycles (e.g., beats) may be displayed via a user interface of an ultrasound system and automatically analyzed. For example, the envelopes of multiple cycles of the displayed Doppler spectrum may be automatically traced via a single input selection initiating the automatic tracing (e.g., via an automatic measurement input), without a user having to manually trace each cycle. This may reduce errors in manual tracing by a user and result in more accurately determined measurement graphics and parameters. Additionally, the automatic tracing my reduce an amount of time required for tracing multiple cycles of Doppler ultrasound data, thereby increasing the efficiency and ease of use of the analysis system. Further, measurement parameters averaged over all traced cycles of the Doppler spectrum may be displayed at the same time as displaying the traced Doppler spectrum. Further still, in response to receiving a selection of an individual traced cycle of the displayed Doppler spectrum, the displayed measurement parameters may be automatically (via the processor) updated to display the measurement parameters for only the individually selected cycle (and not averaged over all traced cycles). As a result, a user may easily and quickly see data for each individually traced cycle and more easily decide which cycles to keep and reject for the final Doppler spectrum data. This may increase the accuracy of diagnosis using the analyzed Doppler spectrum data. Thus, the technical effect of automatically tracing a Doppler spectrum displayed via a user interface of an ultrasound system, the Doppler spectrum including data from a plurality of heart cycles acquired with the ultrasound system; displaying measurement graphics and parameters for the traced Doppler spectrum; receiving a selection of a number of cycles out of the plurality of heart cycles to include in the traced Doppler spectrum; and automatically updating the displayed measurement parameters for the selected number of cycles is to more quickly and easily analyze Doppler spectrum data, thereby reducing analysis time and user errors and therefore increasing an accuracy of diagnosis using the analyzed data.

As one embodiment, a method comprises: automatically tracing a spectrum (e.g., a Doppler spectrum) displayed via a user interface of an ultrasound system, the spectrum including data from a plurality of cycles (e.g., heart cycles) acquired with the ultrasound system; displaying measurement parameters and measurement graphics for the traced spectrum (where the measurement parameters correspond to the measurement graphics); and automatically updating the displayed measurement parameters for a selected number of cycles out of the plurality of cycles to include in the traced spectrum. In one example, the number of cycles selected may be less than a total number of cycles of the plurality of cycles and the automatically updating may include automatically updating the displayed measurement parameters using only the selected number of cycles out of the plurality of cycles to include in the traced spectrum. In one example, displaying the measurement parameters for the traced spectrum includes automatically displaying average measurement parameters that are averaged over all traced cycles of the traced spectrum in response to the automatic tracing. In another example, displaying the measurement parameters for the traced spectrum includes displaying measurement parameters for an individual cycle of the traced spectrum in response to receiving an input selection of the individual cycle. For example, the input selection of the individual cycle may include an input signal indicating that a cursor of a user interface input device is positioned over the individual cycle displayed via the user interface or an input signal indicating that a user has selected the individual cycle via the user interface input device. In one example, the displayed measurement graphics include a boundary trace of the spectrum and the displayed measurement parameters are average measurement parameters including one or more of an average maximum blood flow velocity, an average mean blood flow velocity, an average maximum pressure gradient of the blood flow velocity, an average mean pressure gradient of the blood flow velocity, average velocity time integral, and average envelope time. In another example, the displayed measurement graphics include one or more points on the spectrum and the displayed measurement parameters are average measurement parameters including one or more of average blood flow velocity, average pressure gradient of the blood flow velocity, and average ratios between additional measurement parameters. In yet another example, the displayed measurement graphics include one or more points and one or more lines and the displayed measurement parameters are average measurement parameters including one or more of average blood flow velocity, average pressure gradient of the blood flow velocity, average acceleration or deceleration times of blood flow velocity, average acceleration or deceleration slopes of blood flow velocity, and average ratios between one or more additional measurement parameters. The method may further comprise, in response to receiving a user input approving the selected number of cycles, generating and displaying a report of average measurement parameters averaged over all approved cycles and individual measurement parameters for each of the selected number of cycles. In another example, the method may further comprise receiving an input of the selected number of cycles, where receiving the input includes receiving a first user input selecting a first number of cycles out of the plurality of cycles or a second user input rejecting a second number of cycles out of the plurality of cycles, where the first number of cycles and the second number of cycles equal a total number of cycles in the plurality of cycles. The method may further comprise displaying the spectrum and the measurement graphics on top of the displayed spectrum via a display device of the user interface and wherein displaying measurement parameters for the traced spectrum includes displaying the measurement parameters via the display screen at a same time as displaying the measurement graphics. In one example, automatically tracing the spectrum includes automatically determining a trace of an outline of a signal area of the displayed spectrum without inputs from a user selecting any points of the traced outline. For example, the method may further comprise automatically adjusting the measurement graphics on the displayed spectrum in response to receiving a user input adjusting a sensitivity of the automatic tracing. Additionally, the method may comprise adjusting the displayed measurement parameters in response to adjusting the measurement graphics. In yet another example, the spectrum is one of a continuous wave Doppler spectrum or a pulse wave Doppler spectrum and wherein the spectrum includes blood flow velocity data for the plurality of cycles, the plurality of cycles including a plurality of heart beats.

As another embodiment, a method comprises displaying, via a user interface of an ultrasound system, a Doppler spectrum acquired via the ultrasound system, the Doppler spectrum including data from a plurality of heart cycles; automatically outlining a perimeter of the Doppler spectrum; switching between displaying measurement parameters averaged over all heart cycles of the outlined Doppler spectrum and displaying measurement parameters of an individual heart cycle of the outlined Doppler spectrum; and displaying final measurement parameters averaged over only a subset of cycles of the plurality of heart cycles in response to receiving an input selection of the subset of cycles, the subset being less than the plurality of heart cycles. In one example, the switching between displaying measurement parameters averaged over all heart cycles and displaying measurement parameters of the individual heart cycle is responsive to receiving a user input selecting and deselecting the individual heart cycle via a user interface input device. The method may further comprise rejecting data from the plurality of heart cycles of the Doppler spectrum not included within the subset of cycles and wherein the final measurement parameters are determined from data from only the subset of cycles of the outlined Doppler spectrum. In another example, the displayed final measurement parameters include a plurality of measurement parameters for each individual cycle of the subset of cycles a plurality of measurement parameters averaged over all cycles of the subset of cycles. Additionally or alternatively, the method may further comprise displaying the outline of the perimeter of the Doppler spectrum on top of the displayed Doppler spectrum, via the user interface, and further comprising adjusting the automatic outlining in response to receiving a user input adjusting a sensitivity of the automatic outlining. As one example, the switching between displaying measurement parameters averaged over all heart cycles and displaying measurement parameters of the individual heart cycle includes displaying one of the measurement parameters averaged over all heart cycles and the measurement parameter of the individual heart cycle at a same time as displaying the outlined Doppler spectrum, via the user interface.

As yet another embodiment, an ultrasound system comprises: an ultrasound probe; a user interface; and a controller with non-transitory memory including instructions for: generating a Doppler spectrum from multiple heart cycles of ultrasound data acquired via the ultrasound probe; displaying the generated Doppler spectrum via a display device of the user interface; automatically applying a boundary line to a signal area of the displayed Doppler spectrum, for each heart cycle of the multiple heart cycles; displaying, via the display device, average measurement parameters that are averaged over all heart cycles of the Doppler spectrum and based on the applied boundary line; switching to displaying average measurement parameters of an individual cycle of the Doppler spectrum in response to receiving an input selection of the individual cycle; receiving a selection of a subset of cycles out of the multiple heart cycles of the Doppler spectrum; and displaying average measurement parameters averaged over the selected subset of cycles of the Doppler spectrum. As one example, the displayed average measurement parameters are determined based on data along the applied boundary line of the Doppler spectrum and the input selection and selection of the subset of cycles are received at the controller via a user interface input device of the user interface.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
automatically tracing a spectrum displayed via a user interface of an ultrasound system, the spectrum including data from a plurality of cycles acquired with the ultrasound system;
displaying measurement parameters and measurement graphics for the traced spectrum; and
automatically updating the displayed measurement parameters for a selected number of cycles out of the plurality of cycles to include in the traced spectrum.

2. The method of claim 1, wherein displaying the measurement parameters for the traced spectrum includes automatically displaying average measurement parameters that are averaged over all traced cycles of the traced spectrum in response to the automatic tracing.

3. The method of claim 1, wherein displaying the measurement parameters for the traced spectrum includes displaying measurement parameters for an individual cycle of the traced spectrum in response to receiving an input selection of the individual cycle and, wherein the input selection of the individual cycle includes an input signal indicating that a cursor of a user interface input device is positioned over the individual cycle displayed via the user interface or an input signal indicating that a user has selected the individual cycle via the user interface input device.

4. The method of claim 1, wherein the displayed measurement graphics include a boundary trace of the spectrum and wherein the displayed measurement parameters are average measurement parameters including one or more of an average maximum blood flow velocity, an average mean blood flow velocity, an average maximum pressure gradient of the blood flow velocity, an average mean pressure gradient of the blood flow velocity, average velocity time integral, and average envelope time.

5. The method of claim 1, wherein the displayed measurement graphics include one or more points on the spectrum and wherein the displayed measurement parameters are average measurement parameters including one or more of average blood flow velocity, average pressure gradient of the blood flow velocity, a difference between velocities of two points, a time difference between two points, a slope of a line between two points, and average ratios between additional measurement parameters.

6. The method of claim 1, wherein the displayed measurement graphics include one or more points and one or more lines and wherein the displayed measurement parameters are average measurement parameters including one or more of average blood flow velocity, average pressure gradient of the blood flow velocity, average acceleration or deceleration times of blood flow velocity, average acceleration or deceleration slopes of blood flow velocity, and average ratios between one or more additional measurement parameters.

7. The method of claim 1, further comprising, in response to receiving a user input approving the selected number of cycles, generating and displaying a report of average measurement parameters averaged over all approved cycles and individual measurement parameters for each of the selected number of cycles.

8. The method of claim 1, further comprising receiving an input of the selected number of cycles, wherein receiving the input includes receiving a first user input selecting a first number of cycles out of the plurality of cycles or a second user input rejecting a second number of cycles out of the plurality of cycles, where the first number of cycles and the second number of cycles equal a total number of cycles in the plurality of cycles.

9. The method of claim 1, further comprising displaying the spectrum and the measurement graphics on top of the displayed spectrum via a display device of the user interface and wherein displaying measurement parameters for the traced spectrum includes displaying the measurement parameters via the display screen at a same time as displaying the measurement graphics.

10. The method of claim 8, wherein automatically tracing the spectrum includes automatically determining a trace of an outline of a signal area of the displayed spectrum without inputs from a user selecting any points of the traced outline.

11. The method of claim 9, further comprising automatically adjusting the measurement graphics on the displayed spectrum in response to receiving a user input adjusting a sensitivity of the automatic tracing and further comprising adjusting the displayed measurement parameters in response to adjusting the measurement graphics.

12. The method of claim 1, wherein the spectrum is one of a continuous wave Doppler spectrum or a pulse wave Doppler spectrum and wherein the spectrum includes blood flow velocity data for the plurality of cycles, the plurality of cycles including a plurality of heart beats.

13. A method, comprising:
displaying, via a user interface of an ultrasound system, a Doppler spectrum acquired via the ultrasound system, the Doppler spectrum including data from a plurality of heart cycles;
automatically outlining a perimeter of the Doppler spectrum;
switching between displaying measurement parameters averaged over all heart cycles of the outlined Doppler spectrum and displaying measurement parameters of an individual heart cycle of the outlined Doppler spectrum; and
displaying final measurement parameters averaged over only a subset of cycles of the plurality of heart cycles in response to receiving an input selection of the subset of cycles, the subset being less than the plurality of heart cycles.

14. The method of claim 13, wherein the switching between displaying measurement parameters averaged over all heart cycles and displaying measurement parameters of the individual heart cycle is responsive to receiving a user input selecting and deselecting the individual heart cycle via a user interface input device.

15. The method of claim 13, further comprising rejecting data from the plurality of heart cycles of the Doppler spectrum not included within the subset of cycles and wherein the final measurement parameters are determined from data from only the subset of cycles of the outlined Doppler spectrum.

16. The method of claim 13, wherein the displayed final measurement parameters include a plurality of measurement parameters for each individual cycle of the subset of cycles a plurality of measurement parameters averaged over all cycles of the subset of cycles.

17. The method of claim 13, further comprising displaying the outline of the perimeter of the Doppler spectrum on top of the displayed Doppler spectrum, via the user interface, and further comprising adjusting the automatic outlining in response to receiving a user input adjusting a sensitivity of the automatic outlining.

18. The method of claim 13, wherein the switching between displaying measurement parameters averaged over all heart cycles and displaying measurement parameters of the individual heart cycle includes displaying one of the measurement parameters averaged over all heart cycles and the measurement parameter of the individual heart cycle at a same time as displaying the outlined Doppler spectrum, via the user interface.

19. An ultrasound system, comprising:
an ultrasound probe;
a user interface; and
a controller with non-transitory memory including instructions for:
generating a Doppler spectrum from multiple heart cycles of ultrasound data acquired via the ultrasound probe;
displaying the generated Doppler spectrum via a display device of the user interface;
automatically applying a boundary line to a signal area of the displayed Doppler spectrum, for each heart cycle of the multiple heart cycles;
displaying, via the display device, average measurement parameters that are averaged over all heart cycles of the Doppler spectrum and based on the applied boundary line;
switching to displaying average measurement parameters of an individual cycle of the Doppler spectrum in response to receiving an input selection of the individual cycle;
receiving a selection of a subset of cycles out of the multiple heart cycles of the Doppler spectrum; and
displaying average measurement parameters averaged over the selected subset of cycles of the Doppler spectrum.

20. The ultrasound system of claim 19, wherein the displayed average measurement parameters are determined based on data along the applied boundary line of the Doppler spectrum and wherein the input selection and selection of the subset of cycles are received at the controller via a user interface input device of the user interface.
